(19) United States
(12) Patent Application Publication
(10) Pub. No.: US 2005/0192652 A1
(43) Pub. Date: Sep. 1, 2005

(54) Thermal treatment systems with enhanced tissue penetration depth using adjustable treatment pressures and related methods

(76) Inventors: Iulian Cioanta, Weston, FL (US); Richard Barry Klein, Cary, NC (US)

Correspondence Address: GANZ LAW, P.C., P O BOX 2200, HILLSBORO, OR, 97123, US

(21) Appl. No.: 10/788,735

(22) Filed: Feb. 26, 2004

(51) Int. Cl. A61F 7/00; A61F 7/12

(52) U.S. Cl. 607/105; 607/113

(57) ABSTRACT

Methods, systems and computer program products include pressure monitoring and pressure adjustment devices in closed loop systems which control and adjust the pressure in a closed loop system configured to circulate heated liquid to a treatment balloon and concurrently heats and exerts pressures onto targeted tissue. The systems and methods can adjust the pressure to account for the physiology of the subject and the operational losses due to material relaxation over the duration of the thermal treatment.

### RELATED APPLICATIONS

This invention claims the benefit of co-pending International Application No. PCT/US02/28688, filed on Sep. 9, 2002, and U.S. Provisional Application No. 60/318,556, filed on Sep. 10, 2001, the entire disclosures of which are hereby incorporated by reference and set forth in their entirety for all purposes.

### FIELD OF THE INVENTION

The present invention relates to methods of delivering minimally invasive thermal therapies in a lumen or body cavity of a subject and is particularly suitable for treatment of certain conditions of the prostate.

### BACKGROUND OF THE INVENTION

Conventionally, several types of thermal treatment systems have been proposed to treat certain pathologic conditions of the body by heating or thermally ablating targeted tissue. These thermal treatment systems have used various heating sources to generate the heat necessary to treat or ablate the targeted tissue. For example, laser, microwave, and radio-frequency (RF) energy sources have been proposed to produce the heat which is then directed to the targeted tissue in or around the selected body cavity. Thermal treatment systems have been used to thermally ablate prostate tissue as well as to thermally treat or ablate the tissue of other organs, body cavities, and/or natural lumens.

U.S. Pat. No. 6,216,703 describes certain thermal treatment systems (including microwave energy systems) that can allegedly be used to treat both prostatitis and BPH (benign prostatic hyperplasia). The contents of this patent are hereby incorporated by reference as if recited in full herein. However, BPH and prostatitis, while both disorders of the prostate, are themselves distinct and different conditions and each typically is treated with different treatment strategies and therapies. Additional discussion of prostatitis and suitable treatments is found in co-pending and co-assigned U.S. Provisional Patent Application Ser. No. 60/308,344, entitled, Methods of Treating Prostatitis, the contents of which are hereby incorporated by reference as if recited in full herein.

One particularly successful thermal ablation system known as the Thermoflex® System (available from ArgoMed, Inc., of Cary, N.C.) used to treat BPH ablates the prostate by a thermocoagulation process. This thermal ablation system employs a closed loop liquid or water-induced thermotherapy (WIT) system which heats liquid, typically water, external to the body and then directs the circulating heated water into a treatment catheter. The treatment catheter is inserted through the penile meatus and held in position in the subject prior to initiation of the treatment to expose localized tissue in the prostate to ablation temperatures. The treatment catheter includes an upper end portion which, in operation, is anchored against the bladder neck and an inflatable treatment segment which is held relative to the anchored upper end portion such that it resides along the desired treatment region of the prostate. In operation, the treatment segment expands, in response to the captured circulating fluid traveling therethrough, to press against the targeted tissue in the prostate and to expose the tissue to increased temperatures associated with the circulating liquid, thereby thermally ablating the localized tissue at the treatment site.

As an acceptable alternative to surgery (transurethral resection of the prostate (TURP)), the use of WIT (water-induced thermotherapy) has been shown to be a successful and generally minimally invasive treatment of BPH (benign prostatic hyperplasia). Generally stated, the term “BPH” refers to a condition wherein the prostate gland enlarges and the prostatic tissue increases in density which can, unfortunately, tend to close off the urinary drainage path. This condition typically occurs in men as they age due to the physiological changes of the prostatic tissue (and bladder muscles) over time. To enlarge the opening in the prostatic urethra (without requiring surgical incision and removal of tissue), the circulating hot water is directed through the treatment catheter which is inserted into the penile meatus up through the penile urethra and into the prostate as described above. The treatment segment expands with the hot water held therein to press the inflated treatment segment against the prostate, which then conductively heats and thermally ablates the prostatic tissue. For BPH therapies, the circulating water is typically heated to a temperature of about 60°-62° C. and the targeted tissue is thermally treated for a period of about 35-45 minutes to locally kill the tissue proximate to the urinary drainage passage in the prostate and thereby enlarging the prostatic urinary passage.

The closed loop WIT system and other circulating liquid thermal therapy systems employ components formed of flexible materials such as relatively thin flexible catheters with elastomeric treatment balloons and tubing that can relax over the course of the treatment due to their exposure to conditions associated with the delivery of the therapy (including system pressures and/or heat) when the therapy is administered over relatively long treatment times. Additionally, there can be a physiologic response to the treatment, and the size, resiliency, and/or density of the tissue in the treated region of the prostatic urethra may also alter during the treatment (albeit somewhat differently in different subjects based on individual variation in tissue properties). For example, during ablation treatments, the necrosis of the localized treated tissue about the treatment balloon is such that the tissue in this region effectively shrinks. In the past, to attempt to compensate for this phenomenon, additional amounts of liquid were added in bulk to the closed loop circulating system at one point during the thermal therapy to attempt to boost lost pressure. However, as shown in FIG. 1, after additional liquid was added to the system (shown at time=3-4 minutes on the graph) the pressure did increase as expected, only to decrease relatively quickly. The pressure was measured by a digital transducer located on tubing on the out side (downstream) of the treatment catheter. The graph in FIG. 1 represents pressures (psi) over time (minutes) measured about a 5 cm treatment balloon circulating fluid heated to about 60° C. for a time of about 20 minutes while the treatment balloon was held in foam (a prostate model) to simulate its contact with tissue in a body cavity. The peak in the graph indicates the time at which an additional amount of liquid was added to the closed loop system.

Others have proposed monitoring pressure and using pressure information of the localized tissue for angioplasty procedures to attempt to remove plaque or occlusions from small (and sometimes fragile) lumens. For example, U.S. Pat. No. 4,781,192 to Demer describes monitoring pressure and volume in a balloon dilatation device (which operates by the application of pressure alone without heat). Demer plots balloon expansion on a pressure-volume graph to gain information regarding the nature of the occlusion (such as whether it is brittle, elastic, etc.) to assess whether additional inflation cycles should be carried out. Others have proposed monitoring pressure during thermal therapy so as to control the therapy to minimize applied heat. U.S. Pat. No. 5,496,311 proposes low stress angioplasty dilation methods which use heat and monitors pressure to detect a physiologic response in order to heat and apply pressure under low stress conditions to remove plaque or occluding stenotic material without substantially heating or damaging the underling lumen wall. The contents of these patents are hereby incorporated by reference as if recited in full herein.

There remains a need to provide improved thermal therapy systems, particularly improved circulating fluid thermal treatment systems that can enhance the depth or penetration of the treatment.

### OBJECTS AND SUMMARY OF THE INVENTION

It is an object of the invention to provide minimally invasive thermal treatment systems—which can administer thermal therapies that provide increased tissue necrosis and/or increased penetration depth by adjusting the pressure of the treatment balloon so that the treatment balloon maintains robust or firm positive contact with the proximately positioned tissue with a sufficiently elevated force or pressure (such as at or above about 0.5-3 atm) substantially throughout or during selected portions of a thermal therapy treatment session.

It is another object of the present invention to provide economic circulating liquid closed loop thermal therapy systems having automated pressure monitoring and adjustment capability for promoting thermal treatment penetration depth or other operational enhancements.

These and other objects are satisfied by the present invention, which provides, inter alia, methods, systems, and computer program products that can maintain, increase, or adjust, the pressure in the circulating system so that the dilated or expanded treatment balloon is able to dilate or expand a sufficient outward distance to maintain desired robust contact pressure or force against proximate tissue during the delivery of the thermal therapy. The force or pressure may be selected so as to remain elevated above about 0.5 atm for at least selected portions of the treatment (typically from about 0.75-2 atm) and so as to widen or increase the lumen diameter in the treated region. The pressure may be selected so that it remains substantially constant during all or selected portions of the treatment or so that various pressures are activated at different portions of the treatment cycle. The pressure adjustment can be carried out to compensate for material or component relaxation, operational pressure losses in the system and/or so that it may reduce the heat sink effect attributed to blood circulation in the body and/or increase the penetration depth or volume of necrosis administered via the thermal therapy. In other embodiments, the pressure adjustment may be at least partially controlled by the patient, based on the patient's comfort level.

In certain embodiments, the system is able to monitor pressure in the closed loop system and adjusting the pressure (such as by adding or removing fluid from the circulating fluid path) so that, in response thereto, the treatment balloon adapts to contact and follow the movement of or the physiologic change in the walls of the cavity (as the walls of the cavity shrink or exhibit differing degrees of rigidity or flexibility) and/or to compensate for pressure drop in the system during the thermal therapy procedure.

In other embodiments, the system is able to thermally ablate the targeted tissue in the prostatic urethra to provide a hardened scab, shell or crust of sufficient thickness that it is able to define a sufficiently large opening to allow fluid drainage through the treated portion of the urethra so that it acts as an in situ natural stent having sufficient rigidity to allow fluid drainage despite the edema process by the tissue during and/or post-treatment. The scab or crust can be self-absorbed or naturally disappear or be sloughed as the tissue heals and may be able to reduce the amount of time of, or remove the need for, post-treatment catheterization.

Certain embodiments of the invention are directed to a method of administering a thermal therapy to treat a condition of the prostate using a closed loop thermal treatment system. The method includes inserting a treatment catheter having a liquid circulation path and an expandable treatment balloon in fluid communication therewith into the male urethra of a subject such that the treatment balloon is positioned in the lumen of the prostatic urethra. The prostatic urethra lumen has a wall and a cross-sectional width. The treatment catheter defines a portion of a closed loop thermal treatment system. The treatment balloon is expanded outwardly a distance to cause the treatment balloon to contact the wall of the prostatic urethra and exert pressure onto tissue proximate the prostatic urethra. The tissue surrounding the prostatic urethra is heated by substantially continuously circulating heated liquid through the liquid circulation path and the expanded treatment balloon for a time of at least about 15 minutes so that a thermal therapy is administered to the prostatic urethra. The pressure in the closed loop system is monitored and automatically adjusted based on the pressure determined by the monitoring step to compensate for operational pressure losses in the closed loop system and physiological changes in the tissue proximate the targeted treatment region in the prostatic urethra so that the system maintains at least one selected operating pressure during administration of the thermal therapy. The pressure adjustment can be carried out to compensate the system operation to account for different patient (prostatic) tissue density (patient-to-patient) to thereby deliver a more consistent treatment across a patient population.

Other embodiments are directed to closed loop thermal treatment systems. The system can include a treatment catheter having a circulating liquid inlet channel, a circulating liquid outlet channel, and an expandable treatment balloon in fluid communication with the circulating inlet and outlet channels. The system also includes a pump, a heater, temperature sensors, a pressure sensor operably associated with the treatment catheter and a pressure adjustment device operably associated with the pressure sensor and the treatment catheter. The system also includes a closed loop liquid circulation path adapted to circulate a quantity of liquid therein, the path including connecting tubing extending between the pump and the treatment catheter inlet and outlet channels, the path including the catheter inlet and outlet channels and the treatment balloon. The pressure adjustment device is operably associated with the path. The system also includes a controller operably associated with the pump, heater, pressure sensor, temperature sensors, and pressure adjustment device. The controller has computer program code for (a) activating the pump, the heater, the temperature sensors, the pressure sensor and the pressure adjustment device to substantially continuously circulate heated liquid through the liquid circulation path; and (b) automatically adjusting the pressure in the liquid circulation path to compensate for operational pressure losses over a time of at least about 15 minutes in the treatment system and to account for any physiological changes in the tissue proximate the targeted treatment region in the prostatic urethra so that the system maintains at least one selected operating pressure during administration of the thermal therapy. In certain embodiments, the system is configured to accept user input in situ to set the desired operating pressure(s), and other embodiments a series of increasing pressures are used to apply an increased pressure concurrently with heat at the target site in the body.

Other embodiments of the present invention include methods of treating BPH using a closed loop thermal treatment system. The method comprises: (a) inserting a treatment catheter having a liquid circulation path and an expandable treatment balloon in fluid communication therewith into the male urethra of a subject such that the treatment balloon is positioned in the lumen of the prostatic urethra, the prostatic urethra lumen having a wall and a cross-sectional width, and wherein the treatment catheter defines a portion of a closed loop thermal treatment system; (b) expanding the treatment balloon outwardly a distance to cause the treatment balloon to firmly contact the wall of the prostatic urethra and exert pressure onto tissue proximate the prostatic urethra; (c) heating tissue surrounding the prostatic urethra by substantially continuously circulating liquid heated to at least about 57-62° C. (typically less than about 95° C.) through the liquid circulation path and the expanded treatment balloon for a time of at least about 10-20 minutes so that a thermal ablation therapy is administered to the prostatic urethra; (d) monitoring the pressure in the closed loop system; (e) automatically adjusting the pressure in the closed loop system based on the pressure determined by the monitoring step to compensate for operational pressure losses in the closed loop system and physiological changes in the tissue proximate the targeted treatment region in the prostatic urethra so that the system maintains at least one selected operating pressure during administration of the thermal therapy; and (f) increasing the width of the lumen of the prostatic urethra based on the expanding, heating, and pressure adjusting steps.

Still other embodiments of the present invention are directed to methods of treating BPH, comprising: (a) contacting tissue in the prostatic urethra with a heated fluid filled expanded treatment balloon; and (b) circulating fluid in the treatment balloon to concurrently conductively heat and exert pressure onto the prostatic urethra with sufficient force and temperature to thermally ablate tissue in the prostatic urethra to cause tissue necrosis to a penetration depth of at least about 15-20 mm on average when measured about the circumference of the prostatic urethra lumen.

In certain embodiments, the treatment is carried out to generate a crest about the wall of the lumen of the prostatic urethra, the crust having a sufficient thickness to define a natural stent that can maintain an open passage through the prostatic urethra post-treatment. In particular embodiments, the natural stent is able to maintain a sufficient drainage path even during the edema process attributed to the therapy.

Yet another aspect of the present invention is a method of thermally treating a target region in the body. The method comprises the steps of (a) inserting a treatment catheter into a body lumen; (b) heating liquid external of the subject to above about 40-65° C. (and typically below about 95° C.); (c) circulating the heated liquid in the treatment catheter such that it travels, captured in the treatment catheter, to a target treatment region; (d) exposing the tissue in the targeted region to a temperature of above about 40° C. for a predetermined thermal ablation treatment period corresponding to the heated liquid in the circulating step; (e) insulating non-targeted tissue below the targeted region such that the non-targeted tissue is exposed to a maximum temperature of about 44° C. from contact with the treatment catheter during the circulating step; (f) monitoring the pressure in the system; (g) automatically adding or removing liquid from the circulating system based on the monitoring step. The method may also include the step of directing body fluids to drain through the treatment catheter during the circulating and exposing steps.

The method can be used to treat urinary or prostate disorders or conditions such as prostatitis or BPH or to treat tissues adjacent or proximate a natural body lumen or cavity. In certain particular BPH treatment embodiments, the circulating liquid can be heated to 57°-62° C. or higher external of the subject and directed into the treatment catheter at an inlet temperature of above about 57- 62° C. or higher for at least about 10-20 minutes.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a graph of pressure as a function of time illustrating the pressure drop in the system during an exemplary thermal therapy.

FIG. 2 is a flow chart of operations according to embodiments of the present invention.

FIG. 3A is a schematic illustration of a closed loop thermal treatment system with automated pressure adjustment capability according to embodiments of the present invention.

FIG. 3B is a schematic illustration of a thermal treatment system with the catheter and treatment balloon in position in the prostatic urethra according to certain embodiments of the present invention.

FIG. 4A is a schematic illustration of a low volume closed loop circulating fluid system illustrating pressure sensor placement according to embodiments of the present invention.

FIG. 4B is a graph of pressure over time monitored by various sensors in different locations of a closed loop system and during a thermal treatment according to embodiments of the present invention.

FIG. 4C is a graph of pressure over time monitored by various sensors during a thermal treatment. The sensors are located on the out side of the connecting tubing after (downstream of) the catheter of a closed loop system according to embodiments of the present invention.

FIG. 5A is a schematic illustration of the top of a pressure adjustment device according to embodiments of the present invention.

FIG. 5B is a side section view of the device shown in FIG. 5A.

FIG. 5C is a schematic illustration of the top of an alternate pressure adjustment device according to embodiments of the present invention.

FIG. 5D is a side section view of the device shown in FIG. 5C.

FIG. 5E is a side view of another pressure adjustment device according to embodiments of the present invention.

FIG. 5F is a side view of the device shown in FIG. 5E with the baffle or accordion structure having a compressed length relative to the length shown in FIG. 5E which increases the pressure in the closed loop system.

FIG. 5G is a side view of the device shown in FIG. 5E with the baffle structure having an extending length relative to the length shown in FIG. 5F which decreases the pressure in the closed loop system.

FIG. 6A is a schematic illustration of yet another pressure adjustment device according to embodiments of the present invention.

FIG. 6B is a top view of an open modular housing with a pressure adjustment device incorporated into a circulation path according to embodiments of the present invention.

FIG. 7A is a graph of pressure over time measured in the system during administration of a thermal therapy according to embodiments of the present invention.

FIG. 7B is a graph of pressure in the system over time during administration of a thermal therapy according to embodiments of the present invention.

FIG. 7C is a graph of pressure in the system over time during administration of a thermal therapy according to embodiments of the present invention.

FIG. 7D is a graph of pressure in the system over time during administration of a thermal therapy according to embodiments of the present invention.

FIG. 7E is a graph of pressure in the system over time during administration of a thermal therapy according to embodiments of the present invention.

FIG. 7F is a graph of pressure in the system over time during administration of a thermal therapy according to embodiments of the present invention where a patient may control the pressure under a system defined and controlled maximum pressure.

FIGS. 8A-8C are graphs of the depth of tissue penetration of a thermal therapy into proximate issue depending on the pressure activity of the system according to embodiments of the present invention. FIG. 8A illustrates a system where pressure drops from the initial portion of the treatment to the end. FIG. 8B illustrates that the pressure is held substantially constant and FIG. 8C illustrates that the pressure is increased over the treatment time.

FIGS. 9A and 9B are sectional schematic views of a thermally treated region according to embodiments of the present invention.

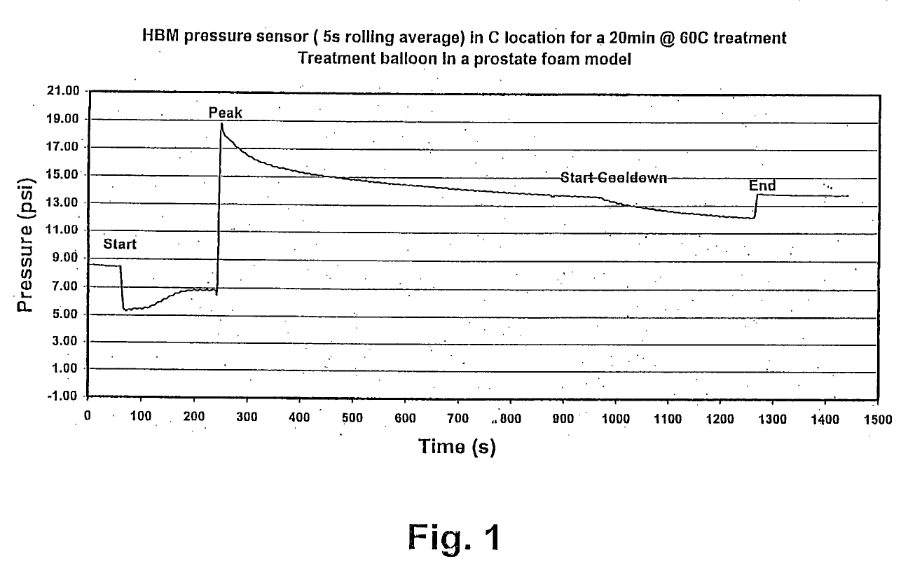

### DETAILED DESCRIPTION OF EMBODIMENTS OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. In the figures, certain elements, regions, or features may be exaggerated for clarity. Like numbers refer to like elements throughout. Also in the figures, broken lines, where used, indicate optional features, operations, or components.

The thermal treatment systems 10 of the present invention may be configured to administer thermal therapies of any desired temperature (cooled and/or heated) in the cavity or natural lumen in the subject's body. For cooling, the thermal treatment systems may be configured to expose the targeted tissue to temperatures below the average body temperature, such as to about 15°-20° C. For heating, the thermal treatment systems can be configured to expose the targeted tissue to temperatures heated to non-ablation temperatures (below about 45° C.) or above ablation temperatures (such as above 45° C.). The present invention finds use for both veterinary and medical applications. The present invention may be advantageously employed for treatment of subjects. “Subjects,” according to the present invention, include animal subjects, and are preferably mammalian subjects (e.g., humans, canines, felines, bovines, caprines, ovines, equines, rodents, porcines, and/or lagomorphs), and are preferably human subjects.

In certain embodiments, the thermal treatment system is a thermal ablation treatment system configured to substantially continuously circulate fluid heated to above about 45° C. (and typically to about 57°-62° C.) for at least a portion of the thermal therapy. Thus, the term “thermal ablation” refers to exposing the targeted tissue to a temperature that is sufficient to kill the tissue. The thermal ablation can be carried out by causing thermocoagulation in targeted tissue via contact with an expandable treatment balloon on a catheter inserted into the subject which is configured to direct circulating hot liquid heated external of the body of the subject to the targeted treatment region within the biological subject.

For ease of discussion, the embodiments of the present invention will be primarily discussed for use in the male urethra. However, the catheters of the present invention may be alternately configured and adapted as appropriate for insertion in other natural lumens or body cavities such as, but not limited to, the colon, the uterus, the cervix, the throat, mouth or other respiratory passages, the ear, the nose, blood vessels, and the like.

In certain embodiments, the thermal treatment systems can be configured to administer thermal ablation therapy to treat BPH or thermal therapies to treat prostatitis. In treating BPH or prostatitis, the walls of the prostatic urethra can be thermally treated by contact with an expandable treatment balloon which expands responsive to the quantity of heated fluid circulating therein, as the fluid travels, captured in the treatment catheter.

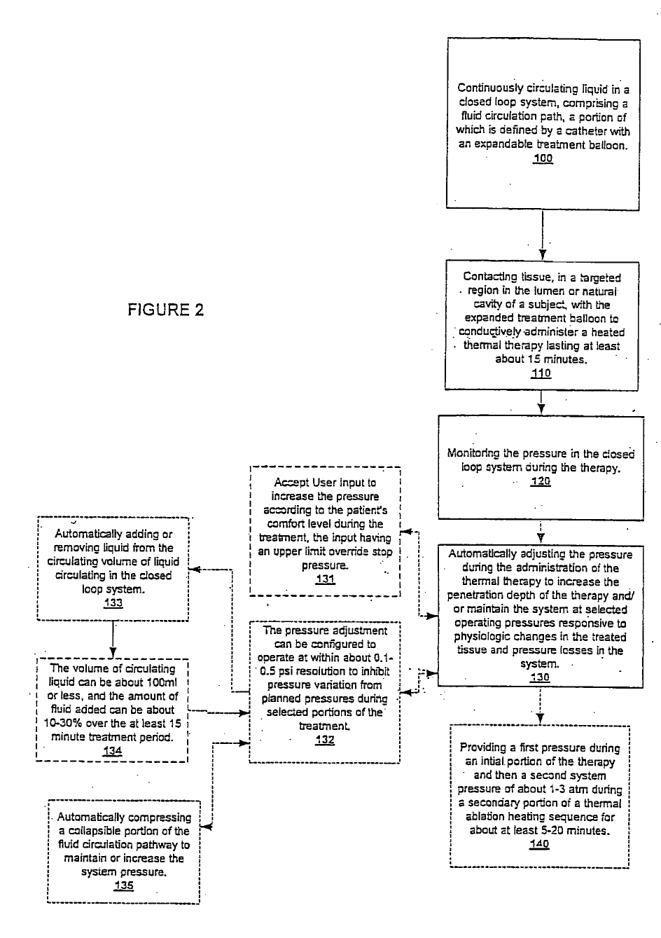

FIG. 2 is a flow diagram of operations of certain embodiments of the present invention. As shown, liquid is substantially continuously circulated in a closed loop system. The closed loop system includes a fluid circulation path, a portion of which is defined by a catheter with an expandable treatment balloon (block 100). Tissue in a targeted region in the lumen or natural cavity of a subject is contacted with the expanded treatment balloon to conductively administer a heated thermal therapy lasting at least about 15 minutes (block 110). The pressure in the closed loop system is monitored (block 120). The pressure is automatically adjusted during the administration of the thermal therapy to increase the penetration depth of the therapy and/or to maintain the system at selected operating pressures responsive to physiologic changes in the treated tissue and pressure losses in the system over the course of the thermal therapy treatment. The system may also be configured to monitor and adjust the temperature of the circulating liquid during the treatment (increasing and/or decreasing over the delivery of the therapy) to administer a concurrent combination of heat and pressure therapy to targeted tissue.

Optionally, for ablation therapies, the operations can be carried out so as to provide a first system pressure during an initial portion of the therapy and then a second substantially constant (or increasing) system pressure of about 0.5-3 atm during a secondary portion of a thermal ablation heating sequence, the thermal ablation lasting at least about 5-20 minutes (block 140). In particular embodiments, the pressure in the system can be at about 0.75-2 atm, and typically at least about 1.0-1.5 atm during at least a latter or secondary portion of the treatment.

In certain embodiments, the system can be configured to accept user input to increase or adjust the pressure to the patient's zone of comfort (block 131). The user input can include a limit or override (either a pressure stop and/or a ramp rate limiter) to assure that the system is not exposed to undue operating pressures. The user input may be accepted during a 5-10 minute initial heating portion of the thermal therapy, and/or during an elevated temperature portion of the thermal therapy (typically administered after about 5-10 minutes).

In certain embodiments, the pressure adjustment can be carried out during the thermal therapy so that the operation is controlled to between about 0.1-0.5 psi resolution to inhibit pressure variation from planned pressures during at least selected portions of the active administration of the thermal therapy treatment (block 132). Maintaining pressures in the system at desired or constant operating pressures by substantially monitoring the system pressure in a manner that can take into account a particular patient's physiology as well as operating conditions may improve consistency between treatments, patient to patient. The pressure adjustment can be carried out by automatically adding or removing liquid from the volume circulating in the closed loop system (block 133) based on the monitored pressure. In certain embodiments, the initial volume of circulating liquid can be on the order of 100 ml or less, and liquid in the additional amount of 10-30% can be added over the at least 15 minute thermal therapy treatment (block 134). In certain embodiments, an initial circulating volume of about 50 ml or less is circulated in the closed loop system; the typical amount of liquid added in over the course of the treatment can be on the order of about 5% or more.

As is known to those of skill in the art, the treatment balloon/catheter used to treat a particular subject can be custom-fit to have a length chosen to fit the length of the patient's prostatic urethra (typically chosen from a range of catheter sizes with treatment balloons ranging in length from about 1.5 cm to about 6 cm). The additional liquid added can be a multiple of the length of the treatment balloon, (i.e., 1.5 ml, 3 ml, or 4.5 ml for a 1.5 cm treatment balloon and 6 ml, 12 ml 15 or 18 ml for a 6 cm treatment balloon).

In other embodiments, a collapsible portion of the fluid circulation pathway can be compressed to maintain or increase the system pressure (block 135).

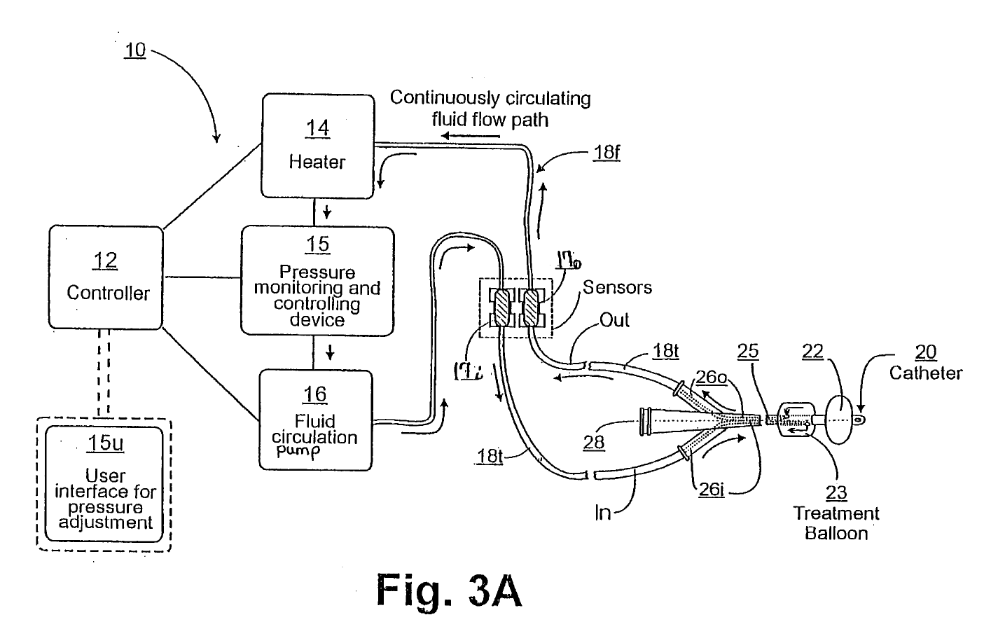

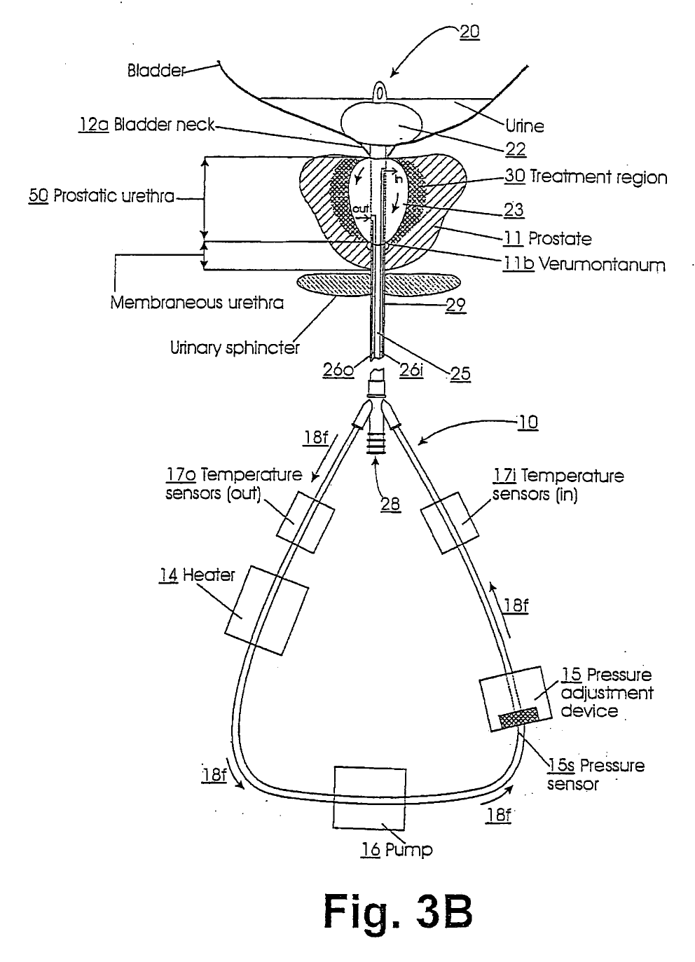

FIG. 3A illustrates one embodiment of a closed loop thermal treatment system 10. As shown, the system 10 includes a controller 12, a heater 14, a 20 pressure monitoring and controlling device 15, a fluid circulation pump 16, a circulating fluid flow path 18f, and a catheter 20 with an expandable treatment balloon 23. The circulating fluid flow path 18f includes a length of elastomeric conduit or tubing 18t extending between the catheter 20 and respective inlet and outlet portions of the circulating fluid flow path 18f. The arrows in the figure indicate the direction of the fluid flow through the system. The components can be arranged in different order and the liquid can flow in the reverse direction. The system 10 can include temperatures sensors 17i, 17o to monitor the liquid temperature as it enters and/or exits the catheter 20.

As shown, the system 10 may optionally include a user interface 15u in communication with the controller 12 to allow a user to adjust the pressure to a custom comfort level. This interface 15u can be a joystick-type peripheral device, a touch screen on a display, a key input or membrane touch switch (such as an arrow) on a keypad, or a voice activated input (“raise” and “lower” or “pressure up” and “pressure down”), or other desired input means. The controller 12 can include means to limit the pressure that the patient can introduce into the system (which may be combined with when the input can be operated), and thus, have a control override to a desired normal range of operation.

In the embodiment shown, the liquid is heated external of the subject (outside the body of the subject) and then introduced to the catheter. In certain embodiments, such as, but not limited to, BPH thermal ablation treatments, the circulating heated fluid can be introduced into the catheter at a temperature of about 45° C.-95° C. for a treatment period which is at least 15-90 minutes in duration, and in particular embodiments heated to a temperature of between about 57-62° C. for about 42-45 minutes in duration.

FIG. 3A illustrates a conventional prior art treatment catheter 20 such as that used in a water induced thermotherapy prostate treatment system identified as the Thermoflex® System available from ArgoMed, Inc. of Cary, N.C. As shown, the treatment catheter 20 includes an anchoring balloon 22, a treatment balloon 23, and an elongated shaft 25. As shown in FIGS. 3A and 3B, the catheter 20 also includes inlet and outlet fluid circulating paths 26i, 26o, respectively, as well as a urinary drainage channel 28 (which can also be used to deliver medicaments therethrough while the catheter 20 is in position in the subject). The anchoring balloon 22 can be in fluid communication with the treatment balloon 23, such that both are inflatable by the circulating heated fluid. Alternately, the anchoring balloon 22 can be fluidly isolated from the treatment balloon 23 (inflatable by a separate air channel directed thereto) (not shown). In this situation, the upper anchoring balloon 22 is separately inflatable and can be inflated before the treatment balloon 23. This can reduce the likelihood that the upper balloon 22 will be inflated below the desired location (potentially introducing damage to the bladder neck 12a or the upper portion of the prostate urethra) and facilitate proper positioning of the catheter 20 in the prostate relative to the bladder. The system 10 can be configured to resist disconnection or to impede the withdrawal of the catheter from the subject until the pressures in the anchoring balloon 22 and the treatment balloon 23 indicate a deflated state.

As shown in FIG. 3B, the treatment can be targeted to a localized treatment region 30 adjacent the prostatic urethra 50, the treatment region 30 being generally described as including the prostatic urethra so as to extend generally below the bladder neck 12a and above the verumontanum 11b of the subject. Alternatively, the treatment region 30 may include the bladder neck 12a or a portion of the bladder neck itself.

It is noted that the circulating heated fluid for thermal ablation treatments can be heated to temperatures above about 45° C. and delivered to the targeted tissue to provide the thermal temperatures for different applications for different lengths of treatment as the desired application dictates. For example, this can be carried out by heating the circulating temperature to at least about 50° C. and then circulating the heated liquid into the catheter, which is positioned in the desired location in the subject so as to expose the targeted tissue to the heated circulating temperature for about 5-90 minutes, and typically about 20-45 or 20-60 minutes.

A suitable thermal treatment system and treatment catheters are available from ArgoMed, Inc. located in Cary, N.C. See also, U.S. Pat. Nos. 5,257,977 and 5,549,559 to Eshel, and co-assigned U.S. patent application Ser. No. 09/433,952 to Eshel et al., the contents of which are hereby incorporated by reference as if recited in full herein.

FIG. 3B also illustrates that the catheter 20 can include a region with increased insulation 29 with respect to other portions of the catheter so as to protect non-targeted tissue from exposure to the circulating heated liquid. The insulated regions 29 can be configured on the catheter as an extra layer or thickness of a material along the proximal or lower shaft portion. Other treatment catheters include a series of circumferentially arranged elongated air channels or conduits which encircle the heated circulating fluid passages and provide thermal insulation along the elongated shaft portion of the catheter as described in U.S. Pat. Nos. 5,257,977 and 5,549,559 to Eshel, the contents of which are hereby incorporated by reference as if recited in full herein. See also, co-pending and co-assigned U.S. Provisional Patent No. 60/248,109, for additional description of suitable catheters, the contents of which are also incorporated by reference as if recited in full herein.

FIG. 3B also illustrates a pressure adjustment device 15 in communication with a pressure sensor 15s in the closed loop system 10. As shown, the pressure sensor 15s can be located external of the body and away from the catheter 20. The pressure adjustment device 15 can be arranged such that it is in-line or offset from the liquid circulation path 15f. Embodiments of the pressure-sensing device 15 will be discussed further below. The travel distance of the circulating liquid can be from about 10-20 feet or more, and is typically about 14-16 feet.

In operation, fluid, which can be water or a water-based liquid, can be heated external of the subject, directed into the catheter 20, and circulated in the enclosed fluid paths 26i, 26o in the catheter 20. The liquid is directed through the shaft 25 via the inlet path 26i to the treatment balloon 23 located proximate the desired treatment site, out of the treatment balloon 23 to the outlet path 26o, and out of the subject. As shown in FIG. 3B, the circulating fluid is directed into the treatment balloon 23, which then expands in response to the quantity of fluid held therein. As shown, temperature sensors 17i, 17o, one 17i positioned on the inlet portion or side of the path 15f (upstream of the catheter), and the other 17o on the outlet portion or downstream side of the path 15f can be used to control the temperature of the circulating liquid. Preferably, a low volume (meaning below about 100 ml, and more preferably below about 50 ml, and still more preferably below about 20 ml) of circulating heated liquid is physically circulated, during operation, at least initially, through the closed loop system 10 to deliver the thermal (or thermal ablation) treatment via the treatment catheter 20. In certain embodiments, water that has been sterilized, distilled, and/or pasteurized can be used as the circulating liquid medium.

In order to anchor the catheter 20 in a desired position or location within the prostate 11 (after the catheter 20 is inserted into the prostate 11) the anchoring balloon 22 is inflated via a fluid introduced through the shaft 25 to the distal portion of the catheter 20 to cause the anchoring balloon 22 to take on an expanded configuration and reside against the bladder neck of the subject. Thus, when expanded, the anchoring balloon 22 is adapted to position the treatment balloon 23 in the prostate relative to the bladder. When deflated, the catheter 20 (including the anchoring and treatment balloons 22, 23) is preferably configured as a smooth, substantially constant profile member to allow for ease of insertion into the body (the balloons may substantially collapse against the central body or shaft of the catheter).

The circulating fluid (and the anchoring balloon inflation media, when separately inflatable) is preferably selected to be non-toxic and to reduce any potential noxious effect to the subject should a situation arise where the balloon integrity may be compromised, accidentally rupture, leak, or otherwise become impaired during service.

The catheter 20 can be flexibly configured so as to be able to bend and flex to follow the shape of the lumen or cavity as it is introduced into the lumen or cavity until a distal portion of the catheter 20 reaches the desired treatment site.

The catheter 20 can be sized as an elongated tubular body with a relatively small cross-sectional area having a thin outer wall so as to be able to be inserted into and extend along a length of the desired lumen to reach the desired treatment site. As used herein, the term “thin outer wall” means a wall having a thickness of about 2 mm or less, and preferably about 1.2 mm or less, and can be in certain embodiments about 0.5 mm or less. For prostate or male urinary applications, the cross-sectional width or outer diameter of the catheter 20 about the tubular body is 20 preferably between about 6-8 mm (18-24 French). Of course, as noted above, the flexible catheter 20 can be alternatively sized and dimensioned to fit other lumens, cavities and/or treatment applications.

In certain embodiments, as shown in FIGS. 3A and 3B a major portion of the cross-sectional area of the shaft region 25 of the catheter 20 is taken up by the size of the fluid channel, or channels, held therein. In certain embodiments, such as, but not limited to, those directed to prostate or male urinary applications, the catheter 20 can include at least three separate fluid channels: the circulating inlet and outlet channels 26i, 26o and the fluid drainage or medicament delivery channel 28 in the shaft region 25.

The flexible catheter 20 can also be configured such that it is sufficiently rigid to be able to maintain an opening in the drainage lumen 28 when inserted and in position in situ (and exposed to increased system pressures of about 0.5-3 atm, and typically at least about 1-2 atm during at least a portion of the thermal therapy) so that the catheter is configured to retain at least about 50% of the cross-sectional area, and preferably at least about 75%-90% or more, of the cross-sectional area, of the drainage lumen 28 relative to the pre-insertion catheter size. As such, the catheter 20 can be flexibly configured such that it is sufficiently conformable to yield to the contours of the subject's body as it is inserted therethrough and into position in the desired region of the subject, yet sufficiently rigid to provide an open drainage lumen when it resides in position in the body (such as in the prostate), and exposed to tissue which is exhibiting distress during or subsequent to undergoing a therapy or thermal treatment.

In certain embodiments, the catheter 20 can be configured such that it is able to maintain a sufficiently sized drainage opening in the drainage lumen 28 to allow desired flow volumes therethrough when exposed to compressive pressures from the treated tissue on the order of about 0.5 atm (7 psi)- 2 atm (28 psi) or 3 atm (42 psi) after exposure to elevated temperatures above about 45° C. for at least about 5-10 minutes, and more preferably for above about 20-30 minutes. The catheters 20 of the instant invention can also be used to maintain an open passage of desired size for other treatments or applications where there is a desire to maintain the open passage in a flexible catheter which is exposed to edema or stress in the subject. See co-pending and co-assigned U.S. Provisional Patent Application No. 60/248,109 for additional description of suitable catheter configurations, the contents of which are hereby incorporated by reference as if recited in full herein.

FIG. 3B illustrates that the system 10 includes at least one pressure sensor 15s in communication with the pressure-adjusting device 15 that is configured to adjust the system pressure responsive to the detected pressure during the delivery of the thermal therapy. The sensor 15s may be positioned in a number of locations along the fluid or liquid circulation path 18f. As shown, the sensor can be located on the system 10 such that it is outside the body of the subject or patient during operation and able to detect system operating pressures which are representative of the pressure at the treatment balloon, as the treatment balloon defines a portion of the liquid circulation path. The pressure adjustment device 15 may be any suitable mechanism, exemplary embodiments of which will be discussed farther below.

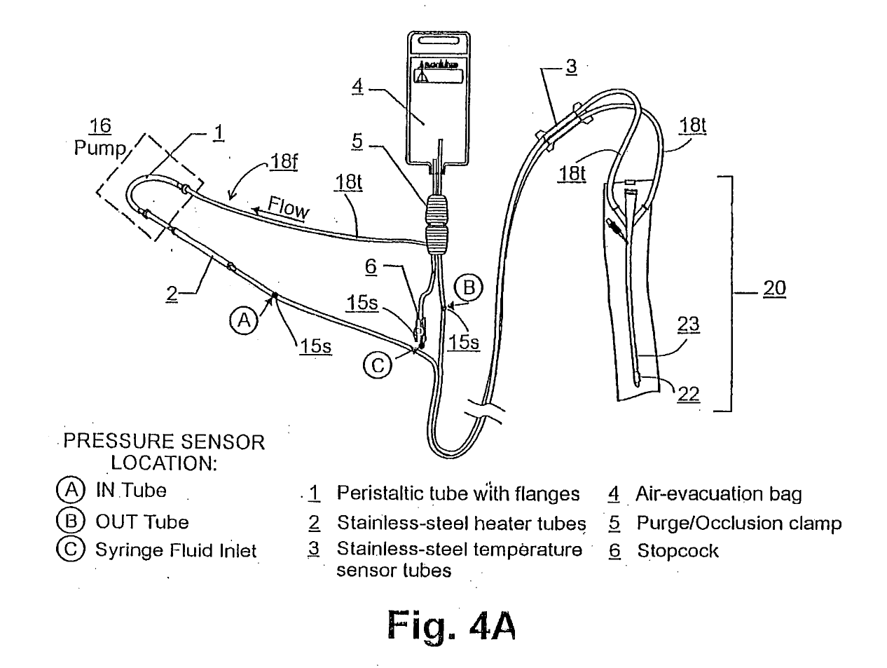

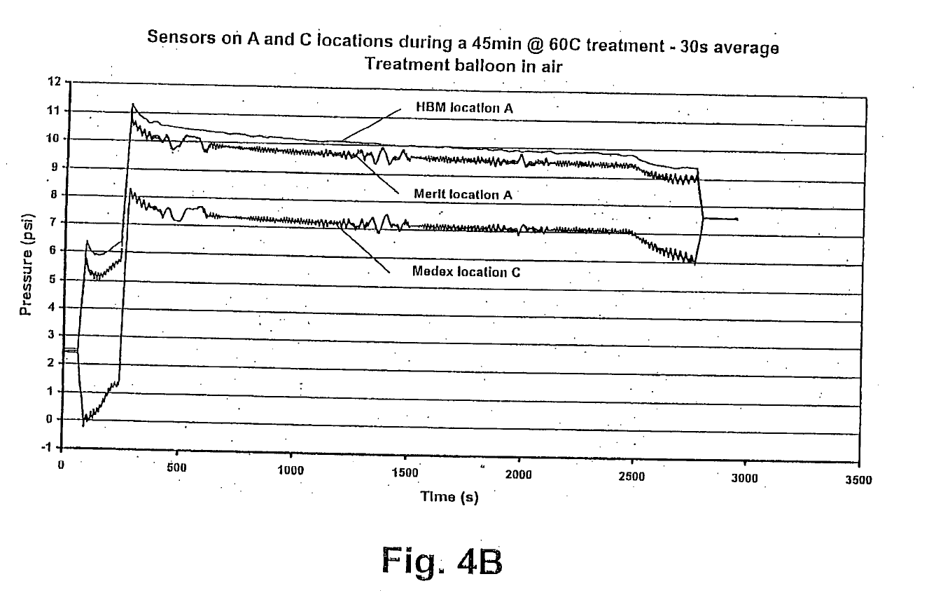

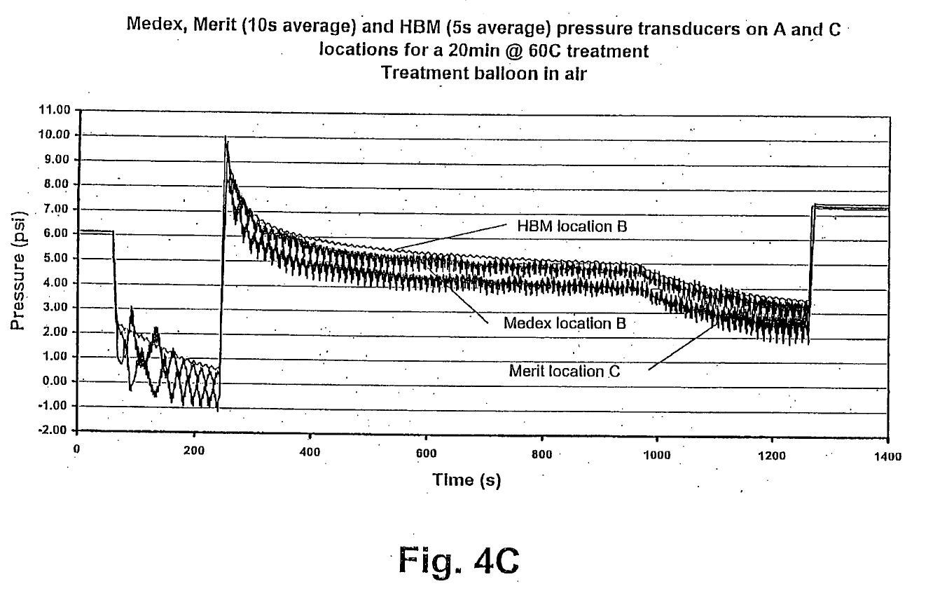

FIG. 4A illustrates a closed loop fluid circulation path 18f with the pump 16 shown in dotted line and other components removed. In this figure, pressure sensors were positioned at three different locations along or in fluid communication with the liquid circulation path 18f. A first sensor is positioned at location “A” along the inlet tube 18t, a second is positioned at location “B” along the outlet tube 18t, and a third is positioned at location “C” at the syringe or fluid inlet port. The pressure sensors 15s can be of any suitable type, such as, but not limited to, transducers similar to those used to measure blood pressure and digital pressure gages. Examples of pressure sensors include the MERITRANS transducer from Merit Medical Systems of South Jordan, Utah, the Medex (MX960) transducer from Medex of Dublin, Ohio, and the Digibar II, PE300, digital pressure gage from HBM GmbH (Hottinger Baldwin Messtechnik) of Germany and similar device identified as model no. DPG1000L-30G from Omega, of Engineering, Inc., of Stamford, Conn. with a pressure range of 0-30 psi and temperature range of 0-70° C.

The sensor 15s in position A is on the tube 18t extending from the heater (not shown) to the catheter treatment balloon 23. In certain embodiments, the tubing 18t can have an inner diameter of about 2-20 mm, and typically about 2.5 mm. The sensor 15s in position B on the outlet tube 18t is positioned in line with the water flowing therethrough. When measured on the out side of the tube (Position B), using the Merit Medical or Medex transducers, the pressure in the balloon appears greater because the fluid is pumped “out” of the (peristaltic) pump 16 which creates a “false” over pressure. In position “A”, because the pump is sucking the fluid at this position in the circulation path 18f, there is an apparent decrease in system pressure.

In the experimental evaluation shown in FIG. 4B, the Merit and Medex transducers used were rated for a compensated pressure range of −10 to 300 mm Hg (maximum design pressure of about 5 psi) but were used to measure up to about 20 psi in the system. In addition, the temperatures used during the evaluation about (60° C.) also exceeded the rated temperature (40° C.). To perform the evaluation, a 5V DC power supply was used along with a Yokogawa MV230 data logger (Yokogawa Electric Corp., Tokyo, Japan) to record the data. Another sensor 15s type used was a digital pressure gage with a digital readout in bars (the HBM model as noted above). The gage was mounted off of a “T” connection with the tubing 18t. The T connection did not appear to constrict flow as its openings were larger than the (2.5 mm) inner diameter of the tubing 18t. The gage readings corresponded to, and thus verified, the results of the other two sensor types, that were operating out of their specification ranges.

As measured, the pressure in the In-tube (location A) was higher than the pressure in the Out-tube (location B). FIG. 4B illustrates data from three sensors (as marked) taken over a 45-minute simulated treatment at 60° C. (with the treatment balloon held in air), two of the lines corresponding to measurements taken by different sensors at location A, and the third line corresponding to measurements taken by a third sensor type at location C. As shown, the data reflects a 30 second rolling average of pressure which smoothes the lines of the graph and acts to reduce the initial peak value. This data is presented as a rolling average, because, in the embodiment shown, the data was gathered using a pulsatile pump to circulate the liquid and the variation in pressures measured in short windows or increments (less than 5 seconds) causes the data to be spiked.

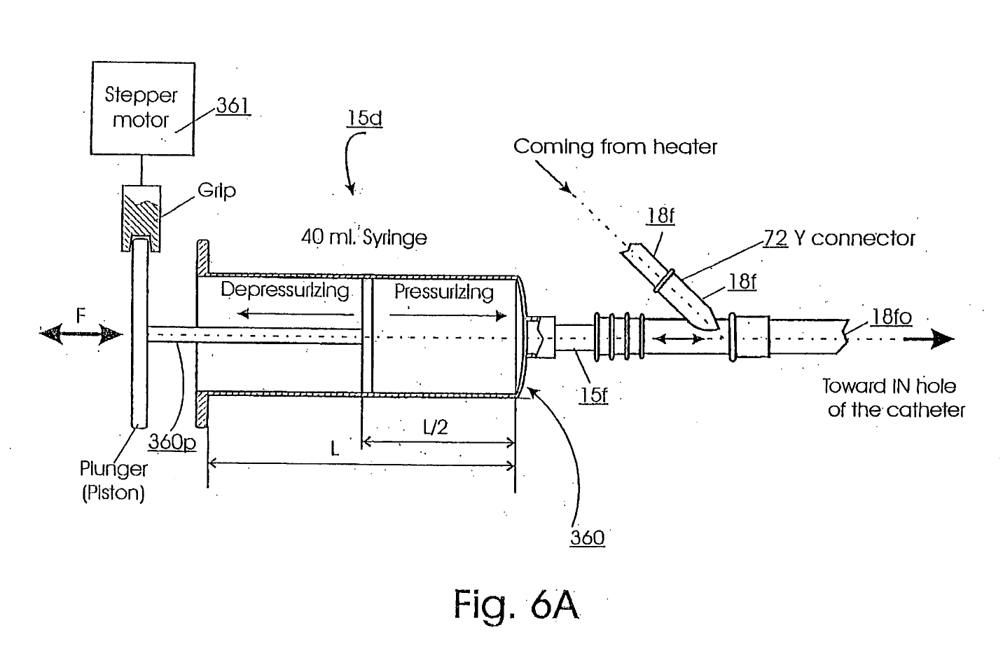

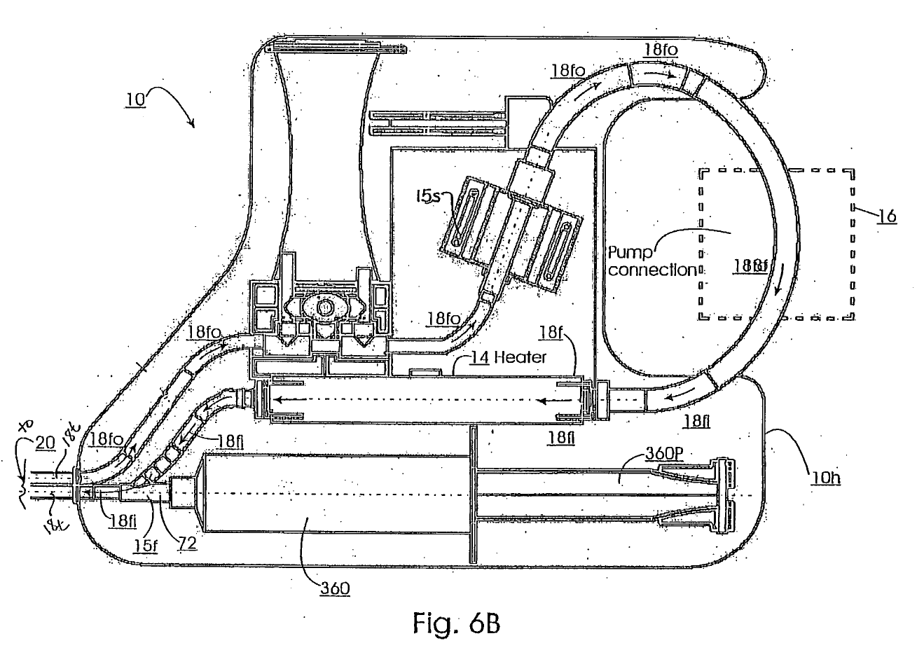

In FIG. 4B, the sensor 15s at location C is a Medex sensor that is positioned such that it is offset from the primary circulation path and in-line with the syringe (location C). This sensor 15s shows an initial pressure drop when the pump turns on while the other sensors indicate initially rising pressures. This is attributed to its location, it is on the downstream side of the catheter before the pump (“18fo”) or the “out” side of the closed loop system 10. Thus, the pressure goes down briefly before it stabilizes (it is suctioned on the “out” side upstream of the pump 18fo and forced “in” on the inlet side 18fi downstream of the pump as shown by FIG. 6B). Note that the pressure in-line with the syringe and offset from the liquid circulation path 18f was only slightly lower than the pressure measured directly from the outlet portion of the system downstream of the catheter 20 and upstream of the pump (location B) and about 3 psi lower than the readings downstream of the pump (location A).

FIG. 4C illustrates data taken over a 20-minute treatment (with the catheter and treatment balloon in air) from location B and location C. The pressure readings correspond, indicating that reliable readings can be obtained by positioning a pressure sensor 15s (transducer) on the out side of the closed loop path 18fo between the pump on one side and the catheter on the other. Depending on where the pressure sensor 15s is positioned, the actual pressures can be determined by compensating the measured value with a calculated adjustment factor. The “real” pressure can be based on a computer program or digital look-up table or equation in a computer software program on the controller 12 (FIG. 3A) or other computer means. In FIG. 4C, the data for the sensors 15s are taken as either 5 second or 10 second rolling averages. In certain conventional systems, a pressure drop of about 4.5-5 psi was indicated over the course of the treatment when measured in air and in a simulated foam model of the prostate). This value corresponds to the peak pressure measured to the pressure at the end of the thermal therapy portion of the treatment (post cool down to end treatment). Another 1-1.5 psi drop may be experienced during a cool down period (typically during the last 5-10 minutes of the treatment).

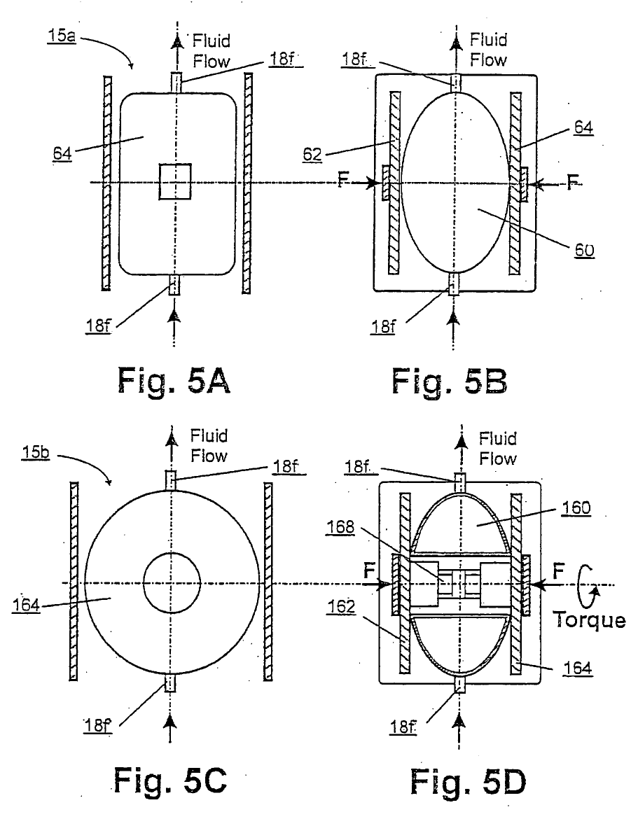

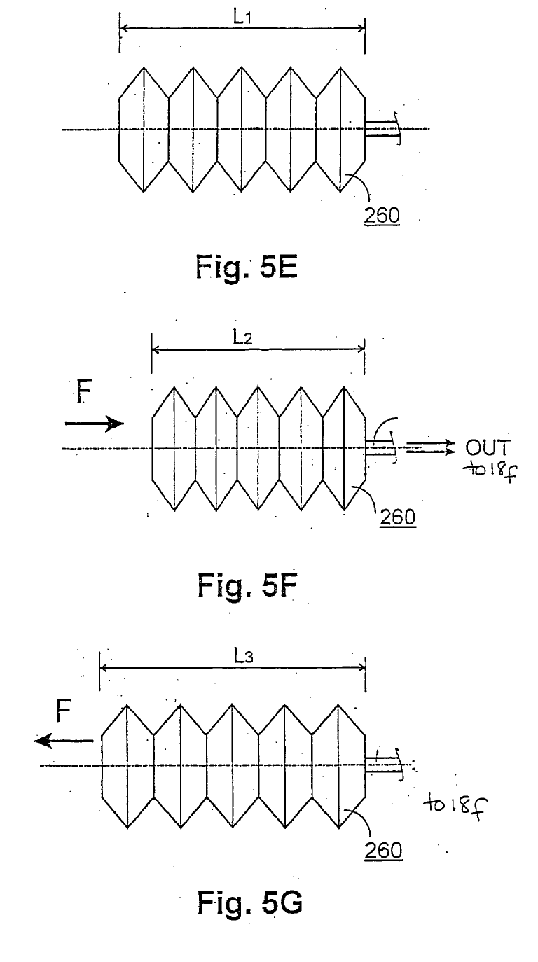

Referring now to FIGS. 5A and 5B, one embodiment of a pressure adjustment device 15a is shown. The device 15a includes a resilient inner member 60 held intermediate of two opposing walls 62, 64. The inner member 60 is configured and formed to be compressible. The inner member 60 can be configured as a bladder or bag or other device or region that has an increased width compared to the width of the flow path 18f that enters and exits therefrom (indicated by the direction of the arrow). In operation, the liquid in the fluid circulation path 18f travels through the inner member 60. The walls 62, 64 are configured to compress the inner member and adjust the pressure in the system 10. In certain embodiments, the walls 62, 64 can be formed such that they are sufficiently rigid to be able to compress the inner member 60. In certain embodiments, the walls 62, 64 are configured as a cooperating pair of plates. The pair includes at least one dynamic plate that can be forced or moved (one or both moved) toward the other in controlled increments by a stepper motor or other mechanism operably associated with the sensor 15s to control (and substantially continuously monitor and adjust) the pressure in the system 10.

In other embodiments, the walls 62, 114 can be stationary and define a portion of an enclosed housing with a fluid inflation chamber sized and configured to surround the inner member 60 therein. A fluid or other inflation source can be controllably directed into the chamber to cause the inner member to compress (or decompress) to adjust the pressure in the system 10. As such, the inner member 60 can be a radially compressible portion of the liquid circulation path 18f.

FIGS. 5C and 5D illustrate another embodiment of a pressure adjustment device 15b. In this embodiment, a cooperating pair of plates 162, 164 are attached by a connecting member 168. The inner member 160 is positioned intermediate the plates 162, 164. In this embodiment, the inner member 160 includes an aperture and the connecting member 168 extends therethrough. The inner member 160 can have an annular, toroidal, or ring-like “donut” configuration. In operation, the connecting member 168 turns to cause one or both of the plates 162, 164 to translate toward or away from the other in controlled increments to compress or release the inner member 160 to thereby adjust the pressure in the system. As before, the inner chamber 160 is compressible and defines a portion of the liquid circulation path 18f such that the liquid in the closed loop system travels therethrough. In certain embodiments, when the inner member 60, 160 is compressed, fluid may exit from the inner member from both ports (in both directions) briefly.

In FIGS. 5E, 5F, and 5G the pressure adjustment device 15c includes an axially compressible bellows or accordion shaped member 260 which is attached to the closed loop circulation path 18f via a Y or T connector (not shown). As such, the bellows member 260 is configured to be offset and in fluid communication with the liquid circulation path 18f such that fluid expelled from the bellows member 260 can be directed into the circulation path via a secondary path connected to the circulation path via the Y or T connector. The bellows member 260 has expandable and compressible segments making its overall length longer (L₃, FIG. 5G) or shorter (L₂, FIG. 5F) depending on the pressure adjustment desired (longer lengths representing lower pressures and shorter lengths representing higher pressures). The compression of the bellows member 260 can be carried out by any desired mechanism so as to control the pressure in the system. FIG. 6A illustrates yet another embodiment of a pressure adjustment device 15d. This embodiment employs a syringe 360 with a quantity of liquid held therein. A plunger or piston 360p is used to direct fluid out of or into the syringe 360 from a supplemental fluid path 15f. As shown, a Y connector 72 defines a junction between the liquid circulation path 18f and the supplemental fluid (adding and removing) path 15f. Other connector or joint types can also be used (such as T's or other configurations). To increase the pressure in the system, additional liquid is injected into the circulation path. Similarly, to decrease the pressure in the system, the liquid can be directed back into the syringe 360. The Y connector 72 can be positioned downstream of the catheter outlet and upstream of the catheter inlet such that the syringe 360 is in fluid communication with the liquid circulation path 18f. In certain particular embodiments, the Y connector 72 and syringe 360 are located downstream of the heater and upstream of the catheter inlet. In certain embodiments, the syringe 360 can be configured to hold between about 30-100 ml, and typically between about 30-50 ml. The handle or arm of the syringe plunger 360p can be connected to a stepper motor 361 which can direct the controlled translation of the plunger and the injection or removal of liquid from the liquid circulation path 18f to maintain or adjust the system 10 to the desired operational pressure. Other suitable control mechanisms can also be used as will be appreciated by those of skill in the art.

FIG. 6B illustrates a portion of a closed loop system 10 in a modular housing 10h with the catheter 20 and certain lengths of conduits between the system 10 and the catheter 20 (not shown). As shown, the liquid circulation path 18f extends from the catheter 20 exit channel to tubing 18t on the out side of the system 18fo into the modular housing 10h and on to the pump 16 where the circulation path 18f transitions to the “in” side of the system 18fi then to the heater 14 and then to exit the housing 10h via tubing 18t to the catheter 20 (inlet channel). In the embodiment shown, the liquid circulation path 18f is joined to the syringe 360 via the Y connector 72 downstream of the pump. The syringe 360 is located in the system 10 so as to be able to introduce additional liquid (or remove liquid) from the circulation path 18f after the liquid travels into a cylindrical heating tube operably associated with the heater 14. The liquid can be preheated in the syringe 360 (by heating elements or by directing air from the heat generating components in the system to flow over the syringe) so as to reduce any heating loss attributed to the introduction of liquid into the system.

The systems or methods may be used to treat BPH, prostatitis, or other urinary or body conditions. For BPH applications, the liquid can be heated external of the body to a temperature in the range of between about 57-62° C. or greater. The circulating heated liquid is directed through the catheter to a treatment balloon such that it travels, captured in the catheter, through the penile meatus, along the penile urethra the bulbous urethra, and the membranous urethra to a localized treatment region in the prostate. The tissue in the localized treatment region in the prostate is exposed to a temperature above about 45° C. for a predetermined thermal ablation treatment period by exposure to the conductive heat from the heated circulating liquid (the liquid can be input at or above about 60° C. for more than about 5-30 minutes, and typically for about 37 minutes). As noted above, the localized treatment region can be the prostatic urethra, leaving the membranous urethra (and the sphincter and penile meatus), non-ablated. This is accomplished in circulating systems (which heat remotely) by insulating the shaft of the treatment catheter up to the treatment balloon to inhibit the exposure of non-targeted tissue to ablation temperatures. Thus, in certain embodiments, the non-targeted tissue is insulated so that it is exposed to a maximum temperature of below about 45° C. from contact with the treatment catheter during the thermal therapy. Additionally, the catheter can be configured to allow urine to drain through the treatment catheter during the procedure.

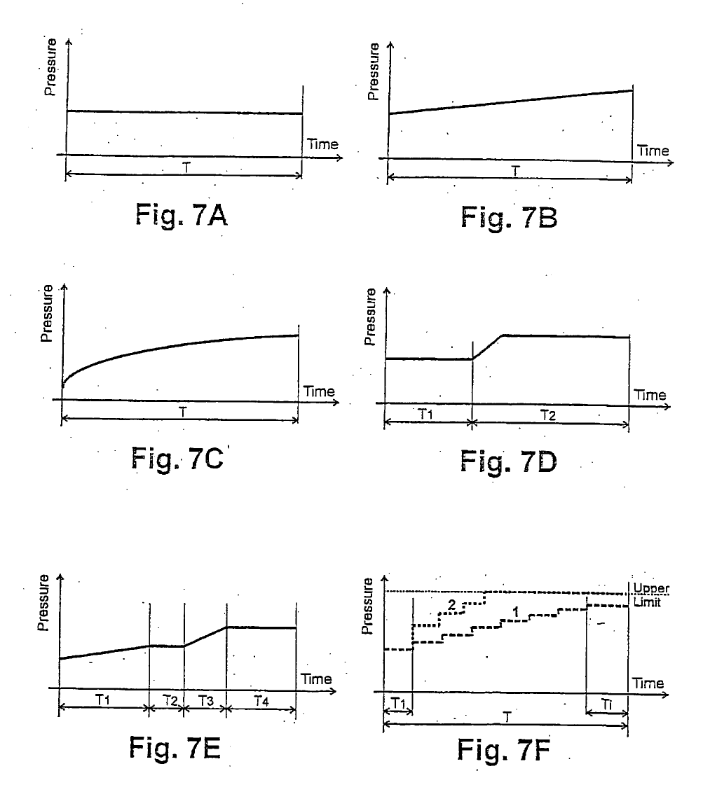

FIGS. 7A-7F illustrate that the thermal therapy can be carried out to increase or maintain the system operating pressure over time (and temperatures can be increased and decreased during the treatment as well as desired). The pressure can be held substantially constant or above certain threshold pressures during a major portion of the ablation treatment time so that the patient is exposed to pressures between about 0.75-2 or 3 atm (which can be carried out with concurrent exposure to ablation temperatures of between about 45-95° C.).

FIG. 7A illustrates that the pressure can be held substantially constant (and elevated) during substantially the entire thermal treatment. FIG. 7B illustrates that the pressure can be gradually increased in a linear manner over the thermal treatment (so that the end of the treatment employs a higher pressure relative to the beginning of the treatment). FIG. 7C illustrates that the pressure can be increased more rapidly during an initial portion of the therapy and then increased more gradually (or held substantially constant) toward the end or a latter portion of the treatment.

FIG. 7D illustrates two sequential treatment periods, an initial period TI, during which a first pressure can be employed. As shown, this initial pressure is less than the next or a second pressure during a second subsequent portion of the treatment (T2), the second pressure can be maintained for a longer portion of the treatment. The first or initial pressure can be concurrently applied to the subject with heat supplied at an initial temperature that is less than a subsequent or second temperature. In certain embodiments, a first lower temperature/lower pressure combination can be used until the subject develops less sensitivity to the treatment (typically after exposed nerves are killed at about 5-10 minutes into the treatment).

FIG. 7E illustrates that the initial pressure can be increased during T1 and held substantially constant during T2 and then increased again during T3 and then held substantially constant during T4 such that the latter portion of the treatment is carried out at higher system pressures than the prior portions. For example, the following sequence of pressures can be used: an initial pressure of about 0.3-0.5 atm ramped over T1 (about 5-10 minutes) to about 0.5-1 atm where it is held during T2 (5-10 minutes), then ramped again during T3 to about 1-2 atm (for about 5-10 minutes), and then held at about 1-2 atm for T4 (about 5-30 minutes). The series of sequentially increasing pressures can be used to deliver the thermal therapy. FIG. 7E illustrates that selected ones of these can be either held substantially constant during that portion of the treatment or gradually ramped or increased during the therapy. This increased pressure can enhance the depth of penetration into the tissue. Setting pressures to predetermined levels can make the treatments more consistent patient to patient irrespective of the physiology of the prostate or the length of the treatment balloon or other variables in the system.

FIG. 7F illustrates that the patient can be allowed to control the administered (system) pressures. The patient may control the pressures during substantially the entire active thermal treatment (T) or at selected portions of the treatment. For example, at an initial T1, or subsequent portion of the treatment, Ti. Patient to patient, the pressure increments may vary depending on the patient's tolerance for pain (shown by the different pressure lines, numbered as “1” and “2”). It is contemplated that when a patient has some control over the procedure, he may be more apt to select or willing to experience greater pressures. The system can be programmed with a safety override that prevents over-pressures from being selected (shown by the upper limit in the figure). In addition, a lower limit can be set so that the patient cannot select non-suitable operating conditions (not shown). The upper and lower limits may be a constant value or can be altered depending on the duration or point in time in the treatment (not shown).

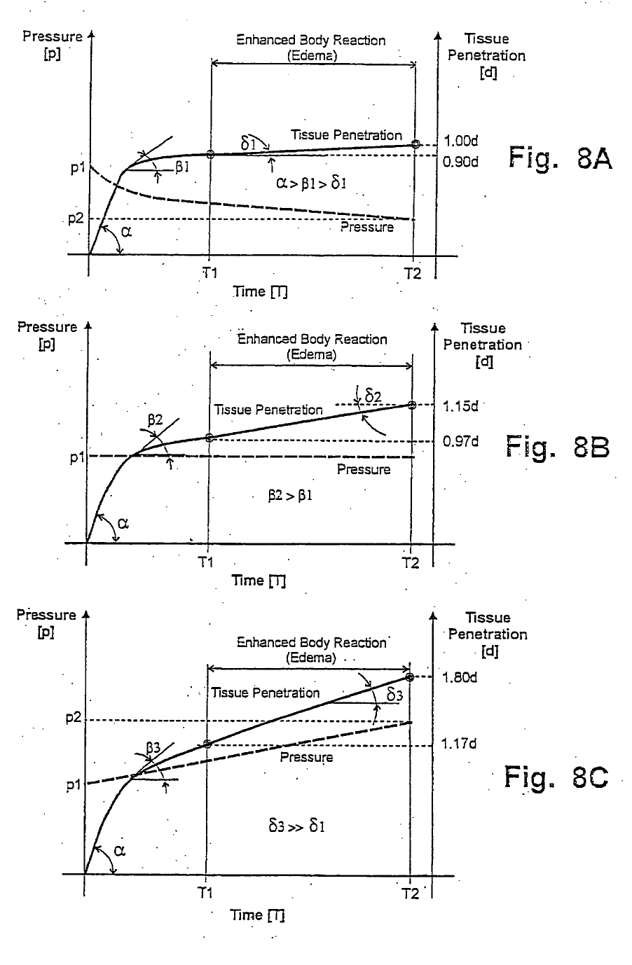

Table 1 provides examples of pressures and temperatures. In the table, where one pressure range/temperature is illustrated it may be administered according to the pressure diagrams of FIGS. 7A, 7B and 7C. Where two times are shown, this can be delivered according to FIGS. 7D-7F. The pressures can be maintained as substantially constant as shown in FIGS. 7A, 8B for all the treatment or selected portions of the treatment as shown in FIG. 7D (T1, T2), FIG. 7E (T2, T4) at which times the temperature may be substantially constant as well or may be increased over the course of the treatment as shown by FIGS. 7B, 8C or during selected portions of the treatment as shown by FIG. 7E (T1, T3), FIG. 7F.

TABLE 1

| P1 | T1 | P2 | T2 | P3 | T3 | P4 | T4 |
|---|---|---|---|---|---|---|---|
| 0.3-1 atm | 40-55° C. | 1-3 atm | 45-95° C. | n/a | n/a | n/a | n/a |
| 0.5-1 atm | 45-50° C. | 1-2 atm | >57-62° C. | n/a | n/a | n/a | n/a |
| 0.5-3 atm | 40-44° C. | n/a | n/a | n/a | n/a | n/a | n/a |
| 0.3-1 atm | 40-50° C. | 0.5-1 atm | 40-57° C. | 1-3 atm | 40-95° C. | 1.5-3 atm | 40-95° C. |

FIGS. 8A-8C graph tissue penetration versus pressure and corresponding thermal ablation treatment times according to embodiments of the present invention. FIG. 8A illustrates that for decreasing pressures during the thermal ablation treatment, the penetration depth is reached at times below about 20 minutes into the treatment (before T1). The latter portion of the ablation treatment may promote body reaction or response to heat damage of the tissue (edema), but may not significantly impact on the depth of penetration into the tissue. That is, about 80-90% of the tissue penetration may occur during the first 10-20 minutes. The slope or angle of penetration provided by the decreasing pressure after the initial penetration is shown as relatively marginal as indicated by “δ₁”. The ablation temperatures may be between about 57°-62° C. or greater (typically below about 95° C.). The pressure at the end of the treatment is shown as p2 and is less than the beginning pressure of p1. The tissue penetration occurs fairly rapidly as is illustrated by the slope of the curve illustrated by “α”. Once the initial penetration depth occurs, a smaller increase and smaller angle of increase is noted (shown by β). As shown, after T1, the increase in tissue penetration is relatively nominal and the angle of the curve is also less (δ). The fight side of the graph illustrates that from time T1 to T2 (which can be on the order of 10-40 minutes), the penetration depth increases slightly from a depth of about 0.9 to a final depth (shown as 1.00 d).

FIG. 8B illustrates that where pressure (p1) is maintained substantially constant during the 10-60 minute ablation treatment, the penetration into the tissue continues to increase during the latter portion of the treatment. This is represented by a larger slope or penetration angle of δ₂ (i.e., δ₂>δ₁). The end penetration value is estimated at 1.15 d over the penetration depth of 0.97 d at time T1.

FIG. 8C illustrates that for a pressure that is substantially continuously increased over the duration of the ablation therapy (shown as from p1 to p2), an even deeper tissue penetration depth can be expected. Similarly, the tissue penetration continues well into the latter portion of the treatment with a penetration slope shown by δ₃ (i.e., δ₃>>δ₁). The penetration depth at T1 may be at about 1.17 d and the end penetration depth at about 1.80 d.

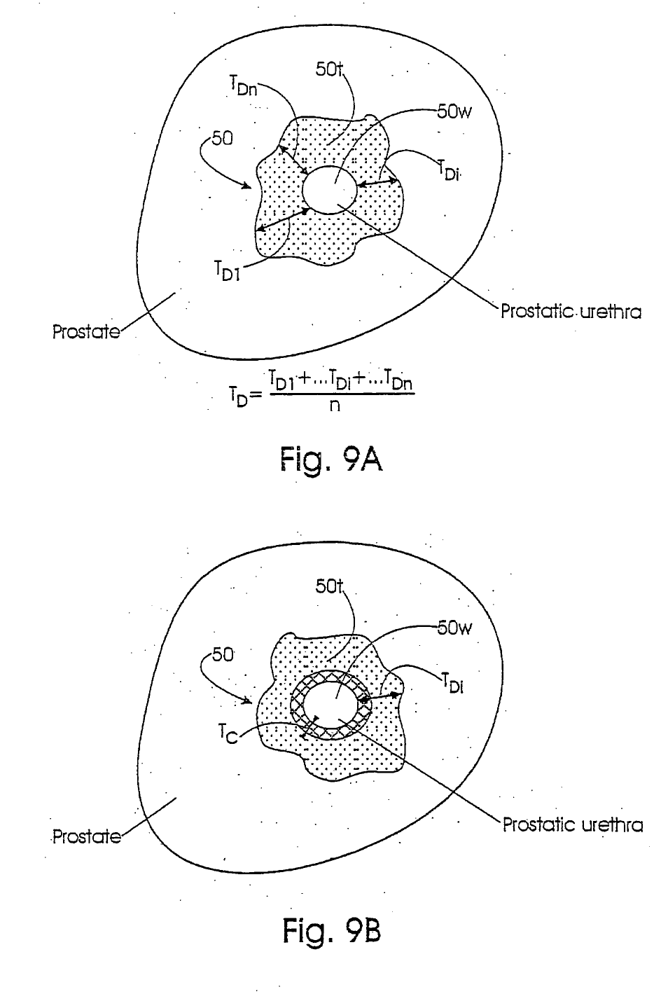

FIGS. 9A and 9B illustrate a sectional view of a lumen and proximate tissue according to embodiments of the present invention. As shown, the prostatic urethra with its lumen 50w and treated tissue 50t has a penetration depth T_{D} about the lumen 50w shown by the crosshatch shading. When measured, on average, the thermal ablation treatment can be carried out to cause tissue necrosis at a penetration depth of at least about 15-20 mm (T_{D}) on average measured about the lumen of the prostatic urethra. Adding a plurality of measurements TD₁ to TDₙ and dividing by the number of measurements “n” can calculate the average TD.

FIG. 9B illustrates that the thermal ablation therapy can be carried out to form a crust or scab of a thickness (Tc) over the wall of the lumen 50w. The crust Tc can be formed such that it has a thickness which is sufficient to define a natural stent. The concurrent heat and pressure ablation treatment can thermally ablate the targeted tissue in the prostatic urethra to provide a hardened scab, shell or crust of sufficient thickness that it is able to define a sufficiently large opening to allow fluid drainage through the treated portion of the urethra so that it acts as an in situ natural stent having sufficient rigidity to allow fluid drainage. The scab or crust can be self-absorbed or naturally disappear or be sloughed as the tissue heals and may be able to reduce the amount of time of, or remove the need for, post-treatment catheterization. In other embodiments, the thermal ablation may have improved penetration depth, but require increased catheterization time due to edema and the like. Nonetheless, the longevity of the treatment itself (i.e., its efficacy) may be improved.

It will be understood that one or more blocks of the block diagram and combinations of blocks in block diagram figures can be implemented or directed to be carried out by computer program instructions. These computer program instructions may be loaded onto a computer or other programmable data processing apparatus to produce a machine, such that the instructions which execute on the computer or other programmable data processing apparatus create means for implementing the functions specified in the flowchart block or blocks. These computer program instructions may also be stored in a computer-readable memory that can direct a computer or other programmable data processing apparatus or associated hardware equipment to function in a particular manner diagrams.

In certain embodiments, the system controller 12 or other operably associated computer device can include computer program code for: (a) activating the pump, the heater, the temperature sensor(s), the pressure sensor and the pressure adjustment device to substantially continuously circulate heated liquid through the liquid circulation path; and (b) automatically adjusting the temperature to desired operational temperatures and automatically adjusting the pressure in the liquid circulation path to compensate for operational pressure losses in the treatment system over a treatment time of at least about 15 minutes and to account for any physiological changes in the tissue proximate the targeted treatment region in the prostatic urethra so that the system maintains at least one selected operating pressure during administration of the thermal therapy.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A method of administering a thermal therapy to treat a condition of the prostate using a closed loop thermal treatment system, comprising:
inserting a treatment catheter having a liquid circulation path and an expandable treatment balloon in fluid communication therewith into the male urethra of a subject such that the treatment balloon is positioned in the lumen of the prostatic urethra, the prostatic urethra lumen having a wall and a cross-sectional width, and wherein the treatment catheter defines a portion of a closed loop thermal treatment system;
expanding the treatment balloon outwardly a distance to cause the treatment balloon to contact the wall of the prostatic urethra and exert pressure onto tissue proximate the prostatic urethra;
substantially continuously circulating heated liquid through the liquid circulation path and the expanded treatment balloon for a time of at least 15 minutes to heat tissue surrounding the prostatic urethra;
monitoring the pressure in the closed loop system; and
automatically adjusting the pressure in the closed loop system based on the pressure determined by the monitoring step to compensate for operational pressure losses in the closed loop system and physiological changes in the tissue proximate the targeted treatment region in the prostatic urethra so that the system maintains at least one selected operating pressure during administration of the thermal therapy.

2. A method according to claim 1, wherein the circulating liquid step comprises heating the circulating liquid to a first temperature during a first portion of the thermal therapy and then heating the circulating liquid to a second, higher temperature during a second portion of the thermal therapy, and wherein the pressure adjusting step is carried out so that the system maintains a substantially constant pressure of between about 0.5 to 3 atm during the second portion of the 30 thermal therapy.

3. A method according to claim 2, wherein the step of adjusting the pressure is carried out so that it maintains a substantially constant system pressure of between about 1-3 atm for the second portion of the administration of the thermal therapy, the second portion of the therapy starting about five-ten minutes from the beginning of the treatment.

4. A method according to claim 1, wherein the cross-sectional width of the lumen of the prostatic urethra increases based on the steps of expanding, circulating liquid, and adjusting the pressure.

5. A method according to claim 1, wherein the administered thermal therapy is a thermal ablation therapy, and wherein the circulating liquid step comprises circulating liquid heated to between about 45°-95° C. for at least about 5-10 minutes.

6. A method according to claim 5, wherein the steps of circulating liquid and adjusting the pressure are carried out such that the closed loop system has a first system pressure and the circulating liquid has a corresponding first heated temperature, and further has a second system pressure with the circulating liquid having a corresponding second heated temperature, the second temperature being generated after about at least five minutes from when the first temperature is generated, wherein the second temperature is greater than the first temperature.

7. A method according to claim 1, wherein the pressure in the system is carried out such that it is substantially constant for a major portion of the duration of the administered thermal therapy.

8. A method according to claim 6, wherein the first temperature is about 45 to 50° C. and the second temperature is between about 57° to 95° C., and wherein the second system pressure is controlled such that it is substantially constant or increases relative to the first system pressure for a time of at least about 5-20 minutes.

9. A method according to claim 5, wherein the steps of expanding, circulating the liquid, and adjusting the pressure are carried out to provide an increased thermal ablation treatment depth sufficient to cause tissue necrosis at a penetration depth of at least about 15 mm on average measured about the lumen 5 the prostatic urethra.

10. A method according to claim 9, wherein the steps of expanding, heating, and adjusting are carried out at times and pressures sufficient to generate a crust about the wall of the lumen of the prostatic urethra, the crust having a sufficient thickness to define a natural stent that can maintain an open passage through the prostatic urethra post-treatment.

11. A method according to claim 1, wherein the liquid circulation path is between about 10 to 20 feet long.

12. A method according to claim 11, wherein the liquid circulation path comprises lengths of interconnected elastomeric tubing with a plurality of connection joints, the tubing being in fluid communication with the treatment catheter.

13. A method according to claim 1, wherein the steps of monitoring and adjusting the pressure are repeated over a plurality of patients having different physiologic prostate densities and using different treatment catheters or different lengths of treatment balloons, and wherein the steps of monitoring and adjusting the pressure are carried out to provide substantially constant system pressures for corresponding portions of the thermal therapies between patients, thereby providing improved consistency of treatment from one patient to another patient.

14. A method according to claim 1, wherein the initial quantity of liquid circulating in the closed loop system is less than 100 ml, and wherein the step of adjusting the pressure comprises introducing additional quantities of liquid therein.

15. A method according to claim 14, wherein the circulation path comprises a resilient portion, and wherein the adjusting step comprises compressing the resilient portion to increase the pressure in the closed loop system.

16. A method according to claim 15, wherein the resilient portion is a compressible bag positioned intermediate two substantially rigid members, and wherein the step of adjusting the pressure is carried out by forcing the two rigid members toward one another.

17. A method according to claim 15, wherein the resilient portion is held in a rigid housing and the adjusting step is carried out by introducing gas into the housing to compress the resilient portion to increase the pressure.

18. A method according to claim 15, wherein the resilient portion is an bellows member which is axially extendable and compressible to greater and lesser axial lengths, and wherein the adjusting step comprises compressing the bellows member to take on a shorter length to increase the pressure in the closed loop system.

19. A method according to claim 1, further comprising accepting user input to set the operating system pressure, constrained by predetermined pressure limits, during at least a portion of the administration of the thermal therapy.

20. A method according to claim 1, wherein the step of adjusting the pressure is carried out by a pressure adjustment device located in-line with the liquid circulation path.

21. A method according to claim 1, wherein the step of adjusting is carried out by a pressure adjustment device located offset to a portion of the liquid circulation path.

22. A method according to claim 1, wherein the thermal therapy is administered to treat prostatitis.

23. A method according to claim 5, wherein the thermal therapy is administered to treat BPH.

24. A closed loop thermal treatment system, comprising:
a treatment catheter having a circulating liquid inlet channel and a circulating liquid outlet channel, and an expandable treatment balloon in fluid communication with the circulating inlet and outlet channels;
a pump operably associated with the treatment catheter;
a heater operably associated with the treatment catheter;
at least one temperature sensor operably associated with the treatment catheter and the heater;
a pressure sensor operably associated with the treatment catheter;
a pressure adjustment device operably associated with the pressure sensor and the treatment catheter;
a closed loop liquid circulation path adapted to circulate a quantity of liquid therein, the path including the treatment catheter inlet and outlet channels and the treatment balloon, wherein the pressure adjustment device is operably associated with the liquid circulation path; and
a controller operably associated with the pump, heater, temperature sensor, pressure sensor, and pressure adjustment device, the controller having computer program code for:
(a) activating the pump, the heater, the temperature sensor, the pressure sensor and the pressure adjustment device to substantially continuously circulate heated liquid through the liquid circulation path; and
(b) automatically adjusting the pressure in the liquid circulation path to compensate for operational pressure losses over a time of at least about 15 minutes in the treatment system and to account for physiological changes in the tissue proximate the targeted treatment region in the prostatic urethra so that the system maintains at least one selected operating pressure during administration of the thermal therapy.

25. A system according to claim 24, wherein the computer code further comprises code for adjusting the operational pressure in the liquid circulating system to predetermined constant or increasing pressures.

26. A system according to claim 24, wherein the computer code further comprises code for adjusting the temperature and for directing the pressure in the liquid circulation path to increase so that it has an increased pressure in a later portion of the treatment over that in the first 5-10 minutes of the treatment.

27. A system according to claim 24, wherein the computer code further comprises code for directing the pressure in the liquid circulation path to remain substantially constant over a major portion of the treatment.

28. A system according to claim 24, wherein the pressure adjustment device comprises a resilient member with a substantially centrally located aperture extending therethrough and opposing rigid members with a threaded coupling member extending therebetween through the aperture of the resilient member, wherein, in operation, the rigid members cooperate to compress the resilient member and increase the pressure in the liquid circulation path and in the treatment balloon.

29. A system according to claim 24, wherein the pressure adjustment device comprises a resilient member encased in a housing having a fluid port, and a fluid source in fluid communication with the fluid port, and wherein, in operation, fluid is directed into the fluid port to compress the resilient member to increase the pressure.

30. A method of treating BPH using a closed loop thermal treatment system, comprising:
inserting a treatment catheter having a liquid circulation path and an expandable treatment balloon in fluid communication therewith into the male urethra of a subject such that the treatment balloon is positioned in the lumen of the prostatic urethra, the prostatic urethra lumen having a wall and a cross-sectional width, and wherein the treatment catheter defines a portion of a closed loop thermal treatment system;
expanding the treatment balloon outwardly a distance to cause the treatment balloon to contact the wall of the prostatic urethra and exert pressure onto tissue proximate the prostatic urethra;
substantially continuously circulating liquid heated to between about 570 to 95° C. through the liquid circulation path and the expanded treatment balloon for a time of at least about 5 minutes to heat tissue surrounding the prostatic urethra so to that a thermal ablation therapy is administered thereto;
monitoring the pressure in the closed loop system;
automatically adjusting the pressure in the closed loop system based on the pressure determined by the monitoring step to compensate for operational pressure losses in the closed loop system and physiological changes in the tissue proximate the targeted treatment region in the prostatic urethra so that the system maintains at least one selected operating pressure during administration of the thermal therapy; and
increasing the width of the lumen of the prostatic urethra based on the expanding, circulating liquid, and pressure adjusting steps.

31. A method according to claim 30, wherein the heating step comprises heating the circulating liquid to a first temperature of between about 45-55° C. during a corresponding first portion of the thermal therapy and then heating the circulating liquid to a second temperature between about 57°-95° C. during a corresponding second portion of the thermal therapy, and wherein the step of adjusting the pressure is carried out so that the system maintains a substantially constant pressure of between about 0.75-3 atm during the second portion of the thermal therapy.

32. A method according to claim 31, wherein the step of adjusting the pressure is carried out so that it maintains a substantially constant system pressure of at least about 1.0-2 atm during the second portion of the thermal therapy, the second portion of the therapy starting about five-ten minutes from the initiation of the treatment.

33. A method according to claim 30, wherein the steps of expanding, circulating liquid, and adjusting are carried out to provide an increased thermal ablation treatment depth sufficient to cause tissue necrosis at a penetration depth of at least about 15-20 mm on average measured about the lumen of the prostatic urethra.

34. A method according to claim 30, wherein the steps of expanding, circulating liquid, and adjusting the pressure are carried out with sufficient heat and pressure to generate a crest about the wail of the lumen of the prostatic urethra having a sufficient thickness to define a natural stent that can maintain an open passage through the prostatic urethra post-treatment.

35. A method according to claim 30, wherein the liquid circulation path is about 10-20 feet long.

36. A method according to claim 30, further comprising insulating a portion of the liquid circulation path to inhibit undue heating of non-targeted tissue.

37. A method according to claim 30, further comprising insulating non-targeted tissue below the targeted region such that the non-targeted tissue is exposed to a maximum temperature of about 44° C. from contact with the treatment catheter during the step of circulating liquid.

38. A method according to claim 37, further comprising directing body fluids to drain through the treatment catheter during the step of circulating liquid.

39. A method of treating BPH, comprising:
contacting tissue in the prostatic urethra with a heated fluid filled expanded treatment balloon; and
circulating fluid to concurrently conductively heat and exert pressure onto the prostatic urethra with sufficient force and temperature to thermally ablate tissue in the prostatic urethra to cause tissue necrosis to a penetration depth of at least about 15-20 mm on average when measured about the circumference of the prostatic urethra lumen.

40. A method according to claim 39, further comprising generating a crest about the wall of the lumen of the prostatic urethra, the crust having a sufficient thickness to define a natural stent that can maintain an open passage through the prostatic urethra post-treatment.

41. A method of thermally treating a target region in the body of a subject using a thermal treatment system with a closed loop circulation path, comprising:
(a) inserting a treatment catheter into a body lumen of the subject;
(b) heating liquid external of the subject to above about 40° C.;
(c) circulating the heated liquid in a closed loop circulation path including a treatment catheter having an expandable treatment balloon;
(d) exposing tissue in a targeted region of the subject to a temperature of above about 40° C. for a predetermined thermal ablation treatment period by heating the tissue based at least in part on the circulating step;
(e) insulating non-targeted tissue below the targeted region such that the non-targeted tissue is exposed to a maximum temperature of about 44° C. from contact with the treatment catheter during the circulating step;
(f) monitoring the pressure in the system; and
(g) automatically adjusting the pressure in the closed loop circulation path to compensate for physiologic changes in the tissue in the targeted region of the subject and pressure decreases over a period of at least about 15 minutes.

42. A method according to claim 41, wherein the step of adjusting the pressure is carried out by removing from or adding to the amount of liquid in the circulation path based on the monitoring step.

43. A method according to claim 42, further comprising directing body fluids to drain through the treatment catheter during the circulating step.
